(19) United States
(12) Patent Application Publication
(10) Pub. No.: US 2003/0087290 A1
(43) Pub. Date: May 8, 2003

(54) Bio-affinity porous matrix in microfluidic channels

(76) Inventors: Michael J. Tarlov, Bethesda, MD (US); Kimberly G. Olsen, Columbia, MD (US); David J. Ross, Silver Spring, MD (US)

Correspondence Address: LARSON & TAYLOR, PLC, 1199 NORTH FAIRFAX STREET, SUITE 900, ALEXANDRIA, VA, 22314, US

(21) Appl. No.: 10/262,904

(22) Filed: Oct. 3, 2002

Related U.S. Application Data
(60) Provisional application No. 60/348,094, filed on Jan. 14, 2002.

(51) Int. Cl. C12Q 1/68; G01N 33/53; G01N 33/542; C12M 1/34

(52) U.S. Cl. 435/6; 435/7.9; 435/287.2

(57) ABSTRACT

A method and device for analyzing species in a sample includes immobilized ligands in a porous matrix disposed in a microchannel. A method of analysis is provided by loading a sample to the microchannel and establishing an analyte flow through the porous matrix. The analyte is detected as the species that binds to the ligand immobilized in the porous matrix. The present method may be adapted to identify multiple species present in a sample by immobilizing different ligands in a series in a single microfluidic channel by sequentially photopolymerization of various porous matrixes, each containing different ligands.

### CROSS-REFERENCE TO RELATED APPLICATIONS

This application claims benefit of the filing date of copending Provisional Patent Application No. 60/348,094, filed on Jan. 14, 2002.

### FIELD OF THE INVENTION

The present invention relates to a method and device for analyzing species in a sample and in particular the present invention relates to the use of a porous matrix disposed in a microfluidic channel for analyzing an analyte present in a sample.

### BACKGROUND OF THE INVENTION

Advances in microchip technology are revolutionizing the field of bioanalytical chemistry. DNA microarrays have taken advantage of many of the benefits of miniaturization, including speed of analysis, smaller sample size, and decreased cost. Many microarray chips have DNA immobilized in a variety of polymeric surface pads in the form of a planar array to facilitate spatially localized detection of DNA hybridization.

Microfluidic channels offer potential analytical advantages over planar arrays, including enhanced mass transfer, lower sample volumes, and ease of integration with miniaturized sample preparation modules. The transfer of DNA detection techniques to microfluidic channels has been limited, however. For example, microfluidic channel walls have been modified to facilitate the detection of proteins. In addition, DNA has also been detected both in solution in microfluidic channels and immobilized onto electrode surfaces by its interaction with tagged liposomes and by surface plasmon resonance spectroscopy.

### SUMMARY OF THE INVENTION

In general, in accordance with the invention, ligands acting as probes are immobilized in a porous matrix disposed in a microfluidic channel thereby forming a microfluidic device. This microfluidic device may be used for analyzing a sample containing an analyte by loading the sample to the microchannel and then flowing the analyte through the porous matrix.

In one implementation of the present invention, acrylamide-modified DNA probes are immobilized in polycarbonate microfluidic channels via photopolymerization in a polyacrylamide matrix. The resulting polymeric, hydrogel plugs are porous under electrophoretic conditions and hybridize with fluorescently-tagged complementary DNA. The double stranded DNA can be chemically denatured and the polycarbonate microfluidic channels with polyacrylamide matrix may be reused with a new analytical sample. The present polymeric structures, i.e., hydrogel plug, provide for immobilizing DNA in microfluidic structures.

In one further implementation, a single microfluidic channel includes hydrogel plugs containing different DNA probe sequences, thereby enabling the selective detection of multiple DNA targets in one electrophoretic pathway.

In another implementation, analyte flow through the porous matrix is generated by applying a pressure gradient to induce flow of fluid and analyte through the pores of the matrix, thereby bringing analyte into contact with the ligands immobilized in the matrix.

In accordance with a further affect of the invention, a method is provided for selectively immobilizing hydrogel plugs of DNA probe/polyacrylamide copolymers in microfluidic channels. The hydrogel plugs, which are photopolymerized in the channels, are permeable to DNA under electrophoretic conditions and hybridize with fluorescently-tagged complementary DNA. The hybridized DNA can be denatured and the DNA hydrogel plug may be reused with a new analytical sample. Using the DNA copolymer plugs, the efficient, directed mass transfer characteristics of microchannels are fully exploited for rapid hybridization of targets present at low concentration and in small sample volumes.

The present invention, in one form thereof, relates to a method for analyzing species in a sample. The method includes supplying a substrate having a microchannel formed therein where the microchannel has a geometry with at least one spatial dimension on the order of micrometers and has a porous matrix disposed in the microchannel. Ligands are immobilized in the porous matrix. A sample containing an analyte is loaded in the microchannel and an analyte flow is established through the porous matrix.

Preferably, the ligand is selected from the group consisting of single stranded DNA, double stranded DNA, catalytic DNA, DNA aptamers, RNA, catalytic RNA, antibodies, antigens, and protein. Advantageously, the method includes the step of detecting the analyte which bonds to the ligand immobilized in the porous matrix.

In a further, alternative embodiment, the ligand includes catalytic nucleic acid and the method further includes allowing the sample to flow through the porous matrix, where the cleaved portion of the catalytic nucleic acid binds to a second ligand immobilized in a second porous matrix.

The present invention, in another form thereof, concerns a method for analyzing species in a sample. The method includes supplying a microchannel having at least some spatial dimension on the order of micrometers and having a porous matrix disposed in the microchannel. One of a plurality of different ligands are immobilized in a different linear section of the porous matrix whereby each different linear section of the porous matrix has immobilized a different ligand that binds a specific analyte. An analyte flow is establishes through the porous matrix.

The present invention, in yet another form thereof, concerns a microfluidic device including a substrate with a microchannel formed therein where the microchannel has a geometry with at least one spatial dimension on the order of micrometers and has a porous matrix disposed in the microchannel. A ligand is immobilized in the porous matrix. In a preferred embodiment, an electrical power supply source is operatively associated with the microchannel for applying an electric current to the microchannel thereby establishing an electrophoretic pathway through the porous matrix. Alternatively, the analyte flow may be pressure-driven. In further advantageous embodiments, the microchannel is formed of a plastic or glass substrate and the porous matrix includes a ligand as an acrylamide-modified ligand in a polyacrylamide matrix.

The invention, in yet another embodiment thereof, concerns a microfluidic device including a microchannel having a geometry with at least one spatial dimension on the order of micrometers and having a porous matrix disposed in the microchannel. A plurality of different ligands are immobilized in a different linear section of the porous matrix whereby each different ligand immobilized in a different linear section of the porous matrix and each different ligand binds a specific analyte when flowed through the porous matrix. In one preferred embodiment, the porous matrix includes a hydrogel plug and may comprises a ligand as a acrylamide-modified ligand in a polyacrylamide matrix.

Further features and advantages of the present invention will be set forth in, or apparent from, the detailed description of preferred embodiments thereof which follows.

### BRIEF DESCRIPTION OF THE DRAWINGS

The invention will now be described in detail with respect to preferred embodiments with reference to the accompanying drawings, wherein:

FIG. 1 is a schematic diagram of a side-by-side two-channel microchannel device according to the present invention;

FIG. 2 is a cross section of the microchannel of FIG. 1 along line 2-2;

FIG. 3(a) to 3(d) depict the concentration of TAMRA tagged DNA oligomer after various periods of electrophoresis, where FIG. 3(a) is after 5 minutes, FIG. 3(b) is after 10 minutes, FIG. 3(c) is after 15 minutes, and FIG. 3(d) is after 20 minutes of electrophoresis; and

FIG. 4(a) is a schematic of a microfluidic device used in the separation of a multiple analyte sample and FIG. 4(b) is an enlargement of a portion of the microfluidic device of FIG. 4(a) according to another embodiment of the present invention.

### DESCRIPTION OF PREFERRED EMBODIMENTS

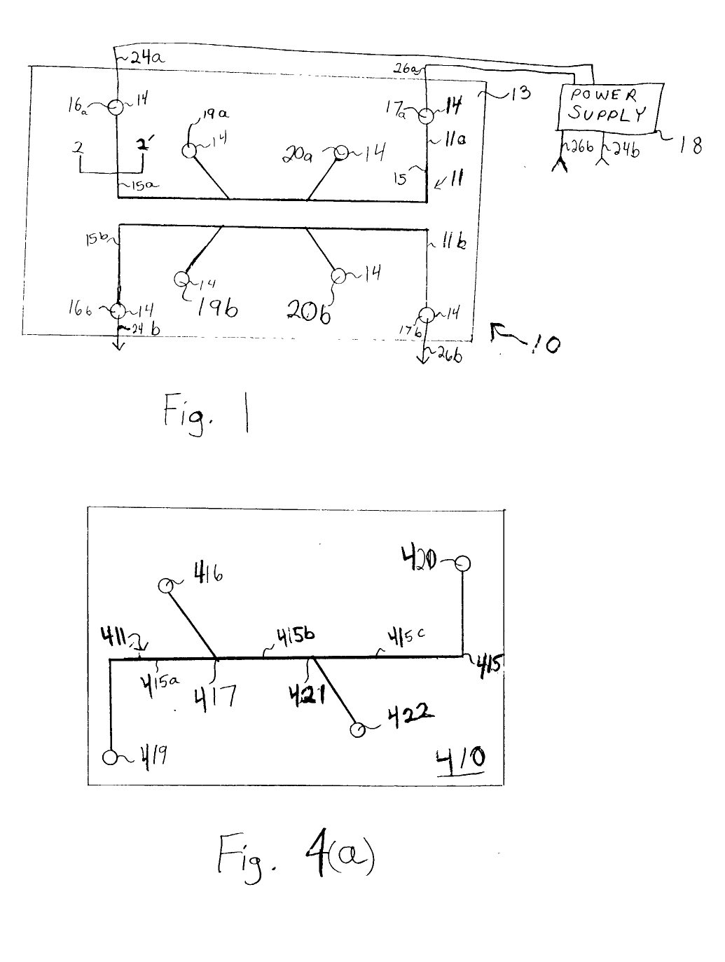

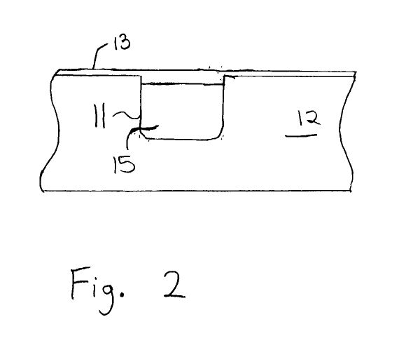

Further description of the present invention is provided with reference to the drawings and in particular to FIGS. 1 and 2 which depicts a plan view of microfluidic device 10 with a plurality of microchannels pathways formed in a substrate 12. The microchannels 11 can be formed in any suitable substrate known in the art. In one embodiment, an excimer laser system is used to form microchannels 11 in a polycarbonate substrate. The dimensions of the microchannels are approximately 50 μM wide and 90 μm deep with a slightly rounded bottom as is best shown in FIG. 2.

The polycarbonate microchannel chip, i.e., substrate 12 is covered with an acrylic lid 13 containing a plurality of 2-mm diameter holes 14 to provide fluid access to the microchannel 11. The two pieces, i.e., substrate 12 and lid 13, were clamped together between glass slides and bonded by placing in a circulating air oven at 103° C. for 30 minutes.

Hydrogel plug 15 is formed in microchannel 11 using a modified procedure similar to the one described by Rehman et al. (Rehman, F. N.; Audeh, M.; Abrams, E. S.; Hammond, P. W.; Kenney, M.; Boles, T. C., Nucleic Acids Research 1999, 27, 649-55) which is directed to fabricating DNA copolymers on optical fibers. Rehman et al. is herein incorporated by reference. Hydrogel plug 15 is a porous matrix into which is incorporated, ligands to be used as a probe for analyzing a sample containing an analyte.

In forming hydrogel plugs 15, the microchannel 11 is filled with a solution containing 0.0006% (w/v) riboflavin, 10% (w/v) 19:1 acrylamide:bis-acrylamide, 10-15 μM acrylamide-modified oligomer, 0.125% (v/v) TEMED, and 0.00007% Fluoresbrite beads in 1×TE (10 mM Tris-HCl, 1 mM EDTA, pH 7.4) buffer, with an equivalent amount of the same solution placed into each of the fluid reservoirs. Fluoresbrite beads were used as visualization markers to minimize fluid flow prior to photopolymerization. The microchannel 11 was illuminated with 515-560 nm light and the emission from the beads was detected at 590 nm. The movement of the Fluoresbrite beads was then monitored while adjusting the volumes of each of the fluid reservoirs and the volumes of each of the fluid reservoirs was adjusted to balance the fluid reservoirs and minimize fluid flow in the microchannel 11. The microchannel 11 was then illuminated with 340-380 nm light focused on a portion of the microchannel 11 for five minutes to effect polymerization. An adjustable aperture in the microscope illumination path was used to define the size of the illuminated spot (typically between 500 μm and 600 μm diameter) and, therefore, the size of the resulting hydrogel plug. After polymerization the photopolymerization solution was rinsed from the open channels on either side of the hydrogel plug 15 and replaced with either a buffer solution containing 0.5 M NaCl and 1×TE (10 mM Tris-HCl, 1 mM EDTA, pH 7.4) buffer or the same buffer containing complementary DNA. The microfluidic device 10 was refrigerated and filled with 0.5 M NaCl and 1×TE (10 mM Tris-HCl, 1 mM EDTA, pH 7.4) buffer when not in use.

Platinum electrodes 24a, 26a are placed in contact with the solution in reservoirs 16a and 17a, respectively, and simultaneously electrodes 24b, 26b are placed in contact with the solution in reservoirs 16b and 17b and all electrodes 24a, 24b, 26a, 26b are connected to a high voltage power supply 18. The current through the microchannel 11 is determined by measuring the voltage drop across a 100 KΩ resistor connected to the power supply in series with the microchannel 11.

The geometry of the microfluidic device 10 permits easy flushing and replacement of solutions in microchannel 11 on both sides of the hydrogel plug 15 after polymerization. In addition, the side-by-side microchannels 11a, 11b facilitate the concurrent photopolymerization of two adjacent microchannels allowing comparison of two channels photopolymerized under the same conditions, but containing different DNA copolymers.

It should be noted that chemical modifications of the microchannel walls is not required before photopolymerization to obtain stable hydrogel plugs. It is believed that the polymers are not chemically bound to the microchannel surface, but nonetheless are able to withstand pressures up to three psi and voltages as high as 100 V for short periods of time. The polymers are stable for extended periods of time to routine exposure to multiple 10-minute applications of 10-25 V. For example, one microfluidic device was utilized for a total of several hours over the course of two days. Advantageously, the exposure period of greater than 25 minutes at a voltage of 10-25 V should be avoided as such conditions could lead to polymer failure.

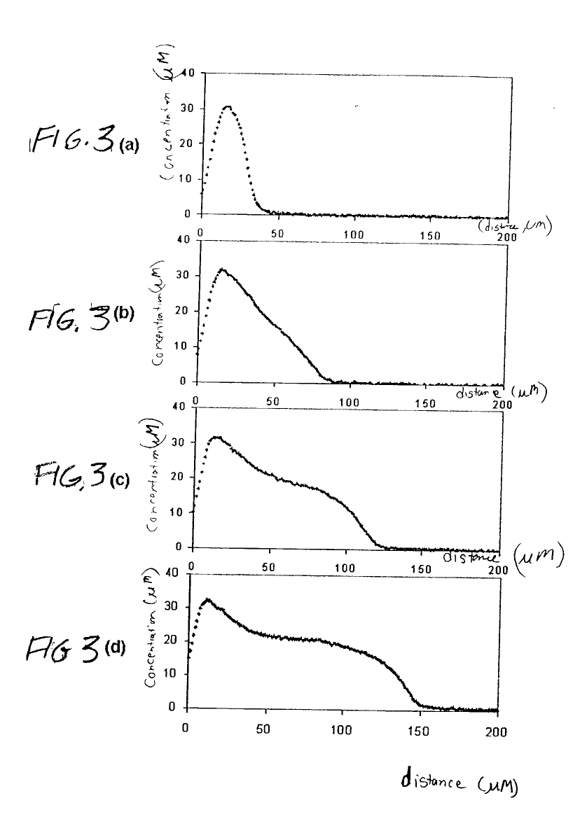

The hydrogel filled microchannel 11 can be used to detect an analyte in a sample. For example, if the hydrogel plug 15 contains single strand DNA (ssDNA), it is possible to use the hydrogel plug 15 (i.e., polymer-filled microfluidic channel 11) to detect complementary DNA via hybridization, as depicted in FIG. 3.

The reproducibility and regeneration of DNA detection of hydrogel plug 15 has been demonstrated. In an example, the hydrogel plug 15a in microchannel 11a contains an immobilized acrylamide-modified 20-base oligomer GCA CCT TGT CAT GTA CCA TC (Seq. ID No. 1) identified as S1, and microchannel 11b holds a hydrogel plug 15b containing a second, different immobilized acrylamide-modified 20-base oligomer AGG CCC GGG AAC GTA TTC AC (Seq. ID No. 2) identified as S2. A 12 μM solution of fluorescein-tagged S1 complement and 0.5 M NaCl in 1×TE buffer was electrophoresed into both hydrogel plugs 15a, 15b and rinsed with 0.5 M NaCl in 1×TE buffer to remove unhybridized DNA. The bound S1 complement was denatured by the electrophoresis of 0.4 M NaOH/0.5 M NaCl in 1×TE buffer through both hydrogel plugs 15a, 15b. The process was repeated in two instances, but in a third, the unbound DNA is removed from the noncomplementary hydrogel plug by inverting the polarity of the electric field for both microchannels 11a, 11b and thereby reversing the movement of the unbound DNA out of the hydrogel plugs 15a, 15b.

In all examples, the hydrogel plug in microchannel 11a contains an immobilized acrylamide-modified 20-base oligomer S1, and the microchannel 11b holds a hydrogel plug 15b containing a second, different immobilized acrylamide-modified 20-base oligomer S2. Initially the wells 16a-19a and 16b-19b and associated segments of the microchannel 11 were filled with 12 μM of the S1 complement tagged with fluorescein in a solution containing 0.5 M NaCl and 1×TE (10 mM Tris-HCl, 1 mM EDTA, pH 7.4) buffer. The wells 20a-17a and 20b-17b and associated segments of the microchannels 11a, 11b were filled with the 0.5 M NaCl-1×TE buffer alone. In the initial example, the complementary DNA solution was electrophoresed into the polymer plug for five minutes at an applied potential of 25 V. Once the hydrogel plugs 15a, 15b appeared to be full of the complementary DNA, the wells 16a-19a, 16b-19b, 20a-17a,20b-17b, and the associated microchannels segments, were all rinsed with 0.5 M NaCl in 1×TE buffer and the clean buffer solution was then electrophoresed through both hydrogel plugs 15a, 15b at 25 V for 5 minutes.

Fluorescence images of the hydrogel plugs indicate that S1 complement remains in the hydrogel plug 15a which contains the immobilized S1 probe, while the S1 complement is largely flushed from the hydrogel plug 15b which contains the noncomplementary S2 probe. The remaining fluorescence at the edges of the hydrogel plug 15b appears slightly different than the fluorescein fluorescence of the hydrogel plug 15a, possibly as a result of riboflavin remaining in the hydrogel plug 15b from the polymerization process.

The hybridization of complementary DNA targets with the probe DNA-containing hydrogel plug is reversible. Duplexes formed in the copolymer can be denatured either electrophoretically or chemically and the hybridization process repeated. For example, if the microchannels 15a, 15b are rinsed with 1×TE buffer containing no NaCl and electrophoresis is re-initiated, a gradual, but incomplete, loss of complement from the hydrogel plugs occurs over a span of 10-15 minutes. A faster and more efficient method for removing the hybridized target DNA is to electrophorese a denaturation solution of 0.4 M NaOH/0.5 M NaCl into the polymer plug at 10 V for 10 min., which removes not only the complement but also the riboflavin remaining from the photopolymerization process.

In the other examples referred to above, the process is essential the same, with one change, namely, the rinsing of the wells 16a-19a, 16b-19b, 20a-17a, 20b-17b and associated segments of microchannels 15a, 15b is eliminated, and the electrophoresis voltage is simply inverted, just as the end of the polymer fills with DNA. Thus voltage inversion reverses the unbound DNA out of the polymer hydrogel plug. Treatment with aqueous NaOH is a commonly used denaturation procedure in Southern blot chemistry, and acrylamide hydrogels are known to be chemically stable under these conditions.

The DNA hydrogel plug 15 also acts to scavenge complementary DNA and low concentrations of complementary DNA in solution can be accumulated and concentrated in the hydrogel plug 15. The ability of the hydrogel plugs 15a, 15b to concentrate DNA is demonstrated by the following example with data presented in FIGS. 3(a) to 3(d).

In FIGS. 3(a) to 3(d), a solution containing 150 nm TAMRA-tagged S2 complement in 0.5 M NaCl in 1×TE buffer was electrophoresed into an S2-containing hydrogel plug. This concentration is sufficiently low such that fluorescence was not observed from the solution in the open channel. However, with continued electrophoresis the TAMRA-tagged S2 complement accumulated in the hydrogel plug over time. Concentration profiles of accumulating TAMRA-tagged S2 complement with increasing time of electrophoresis are shown in FIGS. 3(a) to 3(d).

Concentrations were determined by generating a calibration curve of fluorescence intensity vs. DNA concentration after measuring the fluorescent intensities of solutions of TAMRA-tagged S2 complement of varying known concentrations. Then the fluorescence intensity of the hydrogel plug during the accumulation experiment was compared against the TAMRA-tagged S2 complement calibration curve, resulting in concentration values for the TAMRA-tagged S2 complement hybridizing in the hydrogel plug. The fluorescence was averaged across the entire width of the microchannel 11 for each data point along the length of the hydrogel plug 15. The potential variation in TAMRA fluorescence intensity between solution and the hydrogel plug 15 environments was not considered in calculating concentrations.

The sharp peak of TAMRA-tagged S2 complementary DNA seen on the far left is the accumulation of the complement in the open channel at the solution-plug interface due to the interface acting as an electrophoretic dam. After 25 minutes of electrophoresis, the fluorescence intensity reaches a plateau at a distance of approximately 40 to 100 μm into the plug. The intensity of the plateau roughly corresponds to a concentration of 20 μM in the plug, some two orders of magnitude higher than the initial 150 nM concentration of the solution in the microchannel. Although the exact concentration of the acrylamide-modified ssDNA in the hydrogel plug were not analytically measured, based on the operating conditions, it is calculated that the concentration of ssDNA S2 complement captured is 20 μM based on a hydrogel plug photopolymerization solution containing 15 μM of acrylamide-modified ssDNA and the hydrogel plug which has not been rinsed with 0.5 M NaCl in 1×TE buffer. It is possible to average the fluorescence intensity over a rectangle encompassing the entire hydrogel plug, thereby calculating the average concentration of complementary DNA throughout the entire hydrogel.

It is also instructive to calculate the position of the leading edge of fluorescence along the hydrogel plug, which was defined from concentration profiles like those shown in FIGS. 3(a)-3(d) as the distance from the left edge of the hydrogel at which the concentration first reaches 3 μM. The average concentration of complementary DNA throughout the entire hydrogel plug and the position of the leading edge of fluorescence in the polymer plug, plotted as a function of electrophoresis time, is generally linear. The linear behavior of both the position of the edge and the average concentration indicate that, under these conditions, the rate of capture of DNA targets is limited by the speed at which DNA can be electrophoresed into the plug. The ability of this polymeric system to detect complementary DNA can be considered an integrative process, where sensitivity will depend on the concentration of DNA target, the concentration of DNA probe in the hydrogel, and time allotted for electrophoresis.

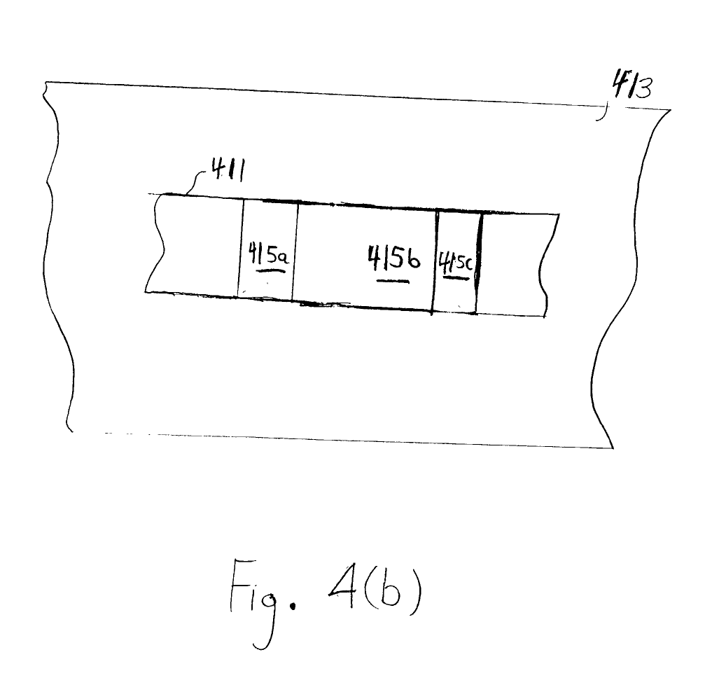

In another embodiment of the present invention, FIG. 4(a) and 4(b) depict a single microchannel fluidic device 410 which can be used for multi-analyte detection. Detection of multi-analytes can be realized by using different color fluorescing tags or by spatially localizing hydrogel plugs that contain different DNA probes. Three spatially-separated hydrogel plugs linear sections, 415a, 415b, 415c, contain different sequence DNA probes or no DNA whatsoever where photopolymerized in the same microchannel 11 but located in a different linear section of the microchannel 411. Hydrogel plug section 415a contains DNA probes complementary to a fluorescein tagged target, S1, while the hydrogel plug section 415c contains DNA probes complementary to a TAMRA tagged target, S2 best shown in FIG. 4(b). The two DNA-containing plugs are separated by hydrogel plug section 415b that does not contain probe DNA.

Initially the entire chip or microfluidic device 410 was filled with a photopolymerization solution containing acrylamide-modified S2, and the hydrogel plug section 415c was created by focusing UV light on the far right portion of the microfluidic channel 411. The wells sections 414, 417, 419 and wells sections 420, 421, 422 and associated sections of the microchannel 411 were rinsed with 0.5 M NaCl in 1×TE buffer and well section 417-421 of the microchannel 411 was rinsed by electrophorescing the 0.5 M NaCl in 1×TE buffer through the section 417-421 of the microchannel 411 with the application of +25 V from reservoir 420 to reservoir 419. The wells sections 420, 421, 422 of the microchannel 411 were then filled with a photopolymerization solution containing no DNA, and the hydrogel plug section 415b was created by electrophorescing the polymerization solution through the hydrogel plug section 415c and into the wells sections 417-421 of microchannel 411 via a +25 V from reservoir 420 to reservoir 419. UV light was then focused onto the center of the section 417-421 of microchannel 411. The rinse and buffer electrophoresis was repeated, and in the third step a photopolymerization solution containing acrylamide-modified S1 was introduced into the well sections 420, 421, 422 of the microchannel 411.

The hydrogel plug section 415a was formed by electrophorescing the polymerization solution through hydrogel plug 415c containing the S2 and hydrogel plug 415b having no DNA and UV light was focused on the far left portion of the microfluidic channel 411. A final rinse of the well sections 414, 417, 419 and well sections 420, 421, 422 of the microchannel 411 with 0.5 M NaCl in 1×TE buffer completes the fabrication process.

To demonstrate multi-analyte detection, a solution containing complements to both S1 and S2 was introduced into the well section 420, 421, 422 of the microchannel 411 and was electrophoresed through all three hydrogel plugs sections 415a, 415b, 415c. After rinsing the well sections 414, 417, 419 and well sections 420, 421, 422 of the microchannel 411 with 0.5 M NaCl in 1×TE buffer and electrophoresis of the buffer through the hydrogel plug sections 415a, 415b, 415c to remove unbound DNA complements at +25 V from reservoir 420 to reservoir 419, green fluorescence is observed predominantly from the hydrogel plug section 415a indicating capture of the S1 complement. Conversely, red fluorescence is observed solely from the hydrogel plug section 415c, indicating the presence of bound S2 complement.

Although DNA has been used herein as an exemplary ligand for use in the present microfluidic device, numerous additional ligands may be employed for use in the present device. A potentially useful list of ligands that could be immobilized in hydrogels for a variety analysis including single stranded DNA for capturing DNA and RNA targets, double stranded DNA for determination of protein-DNA interactions, protein or enzymes for capturing target proteins for proteomic applications, DNA aptamers for capturing target proteins for proteomic applications, and antibodies or antigens for immunoassay applications. Further, catalytic DNA or RNA may be used for analysis of the metal ions, small molecules, metabolites, or proteins.

In the case of catalytic nucleic acid applications, the catalytic DNA or RNA is immobilized in a first hydrogel and an analyte is electrophoresed or pumped through the first hydrogel where the catalytic DNA or RNA undergo self-cleavage. The cleaved strand, having been previously labeled with a fluorophore or suitable group, is then transported to a second hydrogel that contains an immobilized capture strand that is complimentary to the cleaved strand where it is captured. As a result, all cleaved strands are concentrated in the second gel and sensitivity is enhanced.

In addition, multi-analyte detection is also possible using the present device where multiple catalytic DNA's or RNA's are immobilized in a first hydrogel and all are labeled with the same fluorophore. Spatially separated additional hydrogels contain appropriate complimentary sequences which capture cleaved strands. Spatial separation of the captured regions permits the same fluorescent tag to be used for all catalytic reactions.

Further, although previously described herein the analyte is electrokinetically driven through the porous matrix is generated by analyte flow, application of a pressure gradient can be used to induce flow of fluid and analyte through the pores of the matrix, thereby bringing analyte into contact with the ligands immobilized in the matrix.

It will now be apparent to one of ordinary skill in the art that the present invention offers advantages previously not found in the art for use in achieving rapid multiplexed analysis of biological species in applications such as genomics, proteomics, and drug discovery, via biological ligands immobilized in hydrous gel plugs that are contained in microfluidic channels. Further, different ligands can be immobilized in series in a single microfluidic channel by sequential photopolymerization of hydrogel plugs containing the different ligands.

Further, the present invention offers the advantage over prior systems by ensuring that target molecules collide with a captured ligand as the targets are electrophoresed through the hydrogel plugs. The primary advantages of the microfluidic hydrogel plugs versus two-dimensional bio-array formats include a greatly increased capacity relative to two-dimensional formats where monolayers of captured ligands are typically used because they three-dimensional nature of the gel plugs. In addition, the three-dimensional nature of the plugs generally increases the probability that the targets will encounter a captured ligand and biologically bind. Further, mass transport of biological targets to capture ligands is greatly enhanced because all targets are electrophoretically driven through the gel plugs. Therefore, analysis times are greatly reduced. Because the gel plugs are confined to the reduced space of a microchannel, the driving of sample through the plugs results in a concentrating effect. In addition, hydrous plugs containing different ligands can be mobilized in series in microphoretic channels thereby allowing multiplexed detection of targets (i.e., analytes).

Although the invention has been described above in relation to preferred embodiments thereof, it will be understood by those skilled in the art that variations and modifications can be made in these preferred embodiments without departing from the scope and spirit of the invention.

What is claimed is:

1. A method for analyzing species in a sample, said method comprising the steps of:
providing a substrate having a microchannel formed therein, the microchannel having a geometry with at least one spatial dimension on the order of micrometers and having a porous matrix disposed in the microchannel;
immobilizing ligands in the porous matrix;
loading a sample containing an analyte in the microchannel; and
flowing an analyte through the porous matrix.

2. The method of claim 1, wherein the porous matrix comprises a hydrogel plug.

3. The method of claim 2, wherein the hydrogel plug comprises an acrylamide-modified ligand in a polyacrylamide matrix.

4. The method of claim 1, wherein the porous matrix fills the cross section of the microchannel.

5. The method of claim 1, wherein said flowing an analyte step comprises establishing an electrokinetically driven analyte flow.

6. The method of claim 1, wherein said flowing an analyte step comprises establishing a pressure-driven analyte flow.

7. The method of claim 1, wherein the ligand is selected from the group consisting of single stranded DNA, RNA, double stranded DNA, catalytic DNA, catalytic RNA, DNA aptamers, antibodies, antigens, and protein.

8. The method of claim 1, wherein the analyte specifically interacts with the ligand immobilized in the porous matrix.

9. The method of claim 1, wherein the ligand comprises catalytic nucleic acid.

10. The method of claim 9, wherein the cleaved portion of the catalytic nucleic acid binds to a second ligand immobilized in a second porous matrix.

11. A method for analyzing species in a sample, said method comprising the steps of:
providing a microchannel having a geometry with at least one spatial dimension on the order of micrometers and having a porous matrix disposed in the microchannels;
immobilizing one of a plurality of different ligands in a different linear section of the microchannel whereby each different linear section of microchannel has immobilized therein a different ligand;
loading a sample containing an analyte in the microchannel; and
flowing an analyte through the porous matrix.

12. The method of claim 11, wherein the porous matrix comprises a hydrogel plug.

13. The method of claim 12, wherein the hydrogel plug comprises an acrylamide-modified ligand in a polyacrylamide matrix.

14. The method of claim 11, wherein the porous matrix fills the cross section of the microchannel.

15. The method of claim 11, wherein said flowing an analyte step comprises establishing an electrokinetically driven analyte flow.

16. The method of claim 11, wherein said flowing an analyte step comprises establishing a pressure-driven analyte flow.

17. The method of claim 11, wherein the different ligands are selected from the group consisting of single stranded DNA, single stranded RNA, double stranded DNA, catalytic DNA, catalytic RNA, DNA aptamers, antibodies, antigens, and protein.

18. The method of claim 11, wherein one of the analytes specifically interacts with a ligand in one linear section of the microchannel.

19. A microfluidic device, comprising:
a substrate having a microchannel formed therein, said microchannel having a geometry with at least one spatial dimension on the order of micrometers and having a porous matrix disposed in the microchannel; and
a ligand immobilized in said porous matrix.

20. The device of claim 19, wherein said porous matrix comprises a hydrogel plug.

21. The device of claim 20, wherein said hydrogel plug comprises an acrylamide-modified ligand in a polyacrylamide matrix.

22. The device of claim 19, wherein said porous matrix fills the cross section of the microchannel.

23. The device of claim 19, wherein said microchannel is adapted to provide for pressure-driven analyte flow.

24. The microfluidic device of claim 19 further comprising an electrical power source coupled to said microchannel for applying an electric current to the microchannel thereby establishing an electrophoretic pathway through the porous matrix.

25. The microfluidic device of claim 19 wherein said microchannel is formed in one of a plastic substrate and a glass substrate.

26. The microfluidic device of claim 19, wherein said ligand is selected from the group consisting of single stranded DNA, single stranded RNA, double stranded DNA, catalytic DNA, catalytic RNA, DNA aptamers, antibodies, antigens, and protein.

27. The microfluidic device of claim 19, wherein the analyte specifically interacts with the ligand in said porous matrix.

28. A microfluidic device comprising:
a microchannel having a geometry with at least one spatial dimension on the order of micrometers and having a porous matrix disposed in said microchannel; and
a plurality of different ligands, each of said different ligands being immobilized in a different linear section of said microchannel, each of said different ligands specifically interacting with a specific analyte when flowed through said porous matrix.

29. The device of claim 28, wherein said porous matrix comprises a hydrogel plug.

30. The device of claim 28, wherein said hydrogel plug comprises an acrylamide-modified ligand in a polyacrylamide matrix.

31. The device of claim 28, wherein said porous matrix fills the cross section of the microchannel.

32. The device of claim 28, wherein said microchannel is adapted to provide for pressure-driven analyte flow.

33. The microfluidic device of claim 28, further comprising an electrical power source coupled to said microchannel for applying an electric current through said porous matrix and thereby establishing an electrophoretic pathway through said porous matrix.

34. The microfluidic device of claim 28 wherein said microchannel is formed in one of a plastic substrate and a glass substrate.

35. The microfluidic device of claim 28, wherein said ligand is selected from the group consisting of single stranded DNA, single stranded RNA, double stranded DNA, catalytic DNA, catalytic RNA, DNA aptamers, antibodies, antigens, and protein.


SEQUENCE LISTING (2 sequences)

SEQ ID NO: 1 - DNA, 20 bp, Artificial
  artificially generated probe sequence
gcaccttgtcatgtaccatc

SEQ ID NO: 2 - DNA, 20 bp, Artificial
  artificially generated probe sequence
aggcccgggaacgtattcac